(19) United States
(12) Patent Application Publication
(10) Pub. No.: US 2009/0252806 A1
(43) Pub. Date: Oct. 8, 2009

(54) NANOPARTICULATE TACROLIMUS FORMULATIONS

(75) Inventors: Scott JENKINS, Downingtown, PA (US); Gary G. Liversidge, West Chester, PA (US); Elaine Liversidge, West Chester, PA (US)

Correspondence Address: Elan Drug Delivery, Inc. c/o Foley & Lardner, 3000 K Street, N.W., Suite 500, Washington, DC, 20007-5109, US

(73) Assignee: Elan Pharma International Ltd.

(21) Appl. No.: 12/484,073

(22) Filed: Jun. 12, 2009

Related U.S. Application Data
(63) Continuation of application No. 11/300,592, filed on Dec. 15, 2005, now abandoned.
(60) Provisional application No. 60/636,817, filed on Dec. 15, 2004.
(60) Provisional application No. 60/731,869, filed on Nov. 1, 2005.

(51) Int. Cl. A61K 9/14; A61K 31/436

(52) U.S. Cl. 424/490; 424/489; 514/291

(57) ABSTRACT

The present invention is directed to nanoparticulate tacrolimus compositions. The composition comprising tacrolimus particles having an effective average particle size of less than about 2000 nm and at least one surface stabilizer.

### CROSS-REFERENCE TO RELATED APPLICATIONS

This application is a continuation of U.S. patent application Ser. No. 11/300,592, filed on Dec. 15, 2005, which claims the benefit of U.S. Provisional Application No. 60/636,817, filed Dec. 15, 2004, and the benefit of U.S. Provisional Application No. 60/731,869, filed Nov. 1, 2005. The contents of these applications are incorporated herein by reference in their entirety.

### FIELD OF THE INVENTION

The present invention is directed to nanoparticulate compositions comprising tacrolimus. In two exemplary embodiments of the invention, described are injectable nanoparticulate tacrolimus compositions and enteric coated oral dose nanoparticulate tacrolimus compositions, and methods making and using the same.

### BACKGROUND OF THE INVENTION

### Background Regarding Nanoparticulate Active Agent Compositions

Nanoparticulate compositions, first described in U.S. Pat. No. 5,145,684 (“the '684 patent”), are particles consisting of a poorly soluble therapeutic or diagnostic agent having adsorbed onto or associated with the surface thereof a non-crosslinked surface stabilizer. The '684 patent also describes methods of making such nanoparticulate compositions but does not describe compositions comprising tacrolimus in nanoparticulate form. Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract;” U.S. Pat. No. 6,582,285 for “Apparatus for Sanitary Wet Milling;” and U.S. Pat. No. 6,592,903 for “Nanoparticulate Dispersions Comprising a Synergistic Combination of a Polymeric Surface Stabilizer and Dioctyl Sodium Sulfosuccinate;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat. No. 6,745,962 for “Small Scale Mill and Method Thereof;” U.S. Pat. No. 6,811,767 for “Liquid droplet aerosols of nanoparticulate drugs;” and U.S. Pat. No. 6,908,626 for “Compositions having a combination of immediate release and controlled release characteristics;” all of which are specifically incorporated by reference. In addition, U.S. patent application Ser. No. 20020012675 A1, published on Jan. 31, 2002, for “Controlled Release Nanoparticulate Compositions” and WO 02/098565 for “System and Method for Milling Materials,” describe nanoparticulate compositions, and are specifically incorporated by reference.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter” all of which are specifically incorporated herein by reference.

### Background Regarding Tacrolimus

Tacrolimus, or FK-506, is a macrolide immunosuppressant which is reputed to be 100 times more effective than cyclosporine. It is produced by fermentation of Streptomyces tsukubaensis, a monotypic species of Streptomyces. U.S. Pat. No. 4,894,366 and EPO Publication No. 0184162 describe tacrolimus and are herein incorporated by reference in their entirety.

Tacrolimus is sold under the trade name PROGRAF® (available from Fujisawa USA, Inc.) and suppresses some humoral immunity and, to a greater extent, cell-mediated reactions such as allograft rejection, delayed-type hypersensitivity, collagen-induced arthritis, experimental allergic encephalomyelitis, and graft versus host disease. Accordingly, tacrolimus prolongs survival of a host and transplanted graft in animal transplant models of liver, kidney, heart, bone marrow, small bowel and pancreas, lung and trachea, skin, cornea, and limb.

More specifically, experimental evidence suggests that tacrolimus binds to an intracellular protein, FKBP-12. A complex of tacrolimus-FKBP-12, calcium, calmodulin, and calcineurin is then formed, and the phosphatase activity of calcineurin inhibited. This effect may prevent dephosphorylation and translocation of nuclear factor of activated T-cells (NF-AT), a nuclear component thought to initiate gene transcription for the formation of lymphokines (such as interleukin-2, gamma interferon). The net result is the inhibition of T-lymphocyte activation (i.e., immunosuppression).

Tacrolimus has an empirical formula of C₄₄H₆₉NO₁₂·H₂O and a formula weight of 822.05. Tacrolimus appears as white crystals or crystalline powder and is practically insoluble in water, freely soluble in ethanol, and very soluble in methanol and chloroform. Tacrolimus has the following chemical structure:

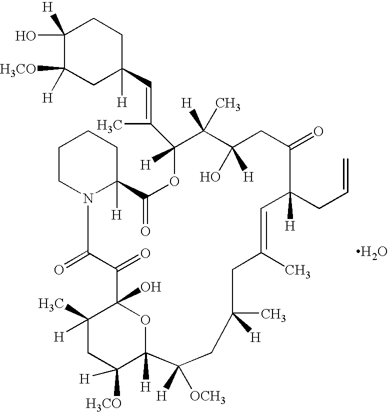

(See, The Merck Index, Twelfth Edition, 9200 (Merck & Co., Inc., Rahway, N.J., 1996).

Absorption of tacrolimus from the gastrointestinal tract after oral administration is incomplete and variable. The absolute bioavailability of tacrolimus is 17±10% in adult kidney transplant patients (N=26), 22±6% in adult liver transplant patients (N=17), and 18±5% in healthy volunteers (N=16).

A single dose study conducted in 32 healthy volunteers established the bioequivalence of the 1 mg and 5 mg capsules. Another single dose study in 32 healthy volunteers established the bioequivalence of the 0.5 mg and 1 mg capsules. Tacrolimus maximum blood concentrations (Cₘₐₓ) and area under the curve (AUC) appeared to increase in a dose-proportional fashion in 18 fasted healthy volunteers receiving a single oral dose of 3 mg, 7 mg, and 10 mg.

In 18 kidney transplant patients, tacrolimus trough concentrations from 3 to 30 ng/mL measured at 10-12 hours post-dose (Cₘᵢₙ) correlated well with the AUC (correlation coefficient 0.93). In 24 liver transplant patients over a concentration range of 10 to 60 ng/mL, the correlation coefficient was 0.94.

With respect to food effects, the rate and extent of tacrolimus absorption were greatest under fasted conditions. The presence and composition of food decreased both the rate and extent of tacrolimus absorption when administered to 15 healthy volunteers. The effect was most pronounced with a high-fat meal (848 kcal, 46% fat): mean AUC and C max were decreased 37% and 77%, respectively; Tmax was lengthened 5-fold. A high-carbohydrate meal (668 kcal, 85% carbohydrate) decreased mean AUC and mean C max by 28% and 65%, respectively.

In healthy volunteers (N=16), the time of the meal also affected tacrolimus bioavailability. When given immediately following the meal, mean Cₘₐₓ was reduced 71%, and mean AUC was reduced 39%, relative to the fasted condition. When administered 1.5 hours following the meal, mean Cₘₐₓ was reduced 63%, and mean AUC was reduced 39%, relative to the fasted condition.

In 11 liver transplant patients, tacrolimus administered 15 minutes after a high fat (400 kcal, 34% fat) breakfast, resulted in decreased AUC (27±18%) and Cₘₐₓ (50±19%), as compared to a fasted state.

Plasma protein binding of tacrolimus is approximately 99% and is independent of concentration over a range of 5-50 ng/mL. Tacrolimus is bound mainly to albumin and alpha-1-acid glycoprotein, and has a high level of association with erythrocytes. The distribution of tacrolimus between whole blood and plasma depends on several factors, such as hematocrit, temperature at the time of plasma separation, drug concentration, and plasma protein concentration. In a U.S. study, the ratio of whole blood concentration to plasma concentration averaged 35 (range 12 to 67).

In patients unable to take oral PROGRAF® capsules, therapy may be initiated with PROGRAF® injection. When considering the uses of PROGRAF® injection, it should be noted that anaphylactic reactions have occurred with tacrolimus injectables containing castor oil derivatives. Therefore, PROGRAF® injection is contraindicated in patients with a hypersensitivity to HCO-60 (polyoxyl 60 hydrogenated castor oil). The initial dose of PROGRAF® should be administered no sooner than 6 hours after transplantation. The recommended starting dose of PROGRAF® injection is 0.03-0.05 mg/kg/day as a continuous IV infusion. Adult patients should receive doses at the lower end of the dosing range. Concomitant adrenal corticosteroid therapy is recommended early post-transplantation. Continuous intravenous (IV) infusion of PROGRAF® injection should be continued only until the patient can tolerate oral administration of PROGRAF® capsules.

PROGRAF® injection must be diluted with 0.9% Sodium Chloride Injection or 5% Dextrose Injection to a concentration between 0.004 mg/mL and 0.02 mg/mL prior to use. Diluted infusion solution should be stored in glass or polyethylene containers and should be discarded after 24 hours. The diluted infusion solution should not be stored in a PVC container due to decreased stability and the potential for extraction of phthalates. In situations where more dilute solutions are utilized (e.g., pediatric dosing, etc.), PVC-free tubing should likewise be used to minimize the potential for significant drug adsorption onto the tubing. Parenteral drug products should be inspected visually for particulate matter and discoloration prior to administration, whenever solution and container permit. Due to the chemical instability of PROGRAF® in alkaline media, PROGRAF® injection should not be mixed or co-infused with solutions of pH 9 or greater (e.g., ganciclovir or acyclovir).

If IV therapy is necessary, conversion from IV to oral tacrolimus is recommended as soon as oral therapy can be tolerated. In a patient receiving an IV infusion, the first dose of oral therapy should be given 8-12 hours after discontinuing the IV infusion. The recommended starting oral dose of Tacrolimus capsules is 0.10-0.15 mg/kg/day administered in two divided daily doses every 12 hours. Co-administered grapefruit juice has been reported to increase tacrolimus blood trough concentrations in liver transplant patients. Dosing should be titrated based on clinical assessments of rejection and tolerability.

There is currently a need for tacrolimus formulations that have enhanced solubility characteristics which, in turn, provide enhanced bioavailability upon administration to a patient, as well as reduced fed/fasted absorption variability. The present invention satisfies these needs by providing methods and compositions comprising a nanoparticulate formulation of tacrolimus. Such formulations include injectable nanoparticulate formulations of tacrolimus that eliminate the need to use polyoxyl 60 hydrogenated castor oil (HCO-60) as a solubilizer, and enteric coated nanoparticulate formulations of tacrolimus. Nanoparticulate tacrolimus compositions are desirable because with a decrease in particle size, and a consequent increase in surface area, a composition is rapidly dissolved and absorbed following administration.

### SUMMARY OF THE INVENTION

The present invention is directed to tacrolimus formulations comprising nanoparticulate tacrolimus having an effective average particle size of less than about 2000 nm and at least one surface stabilizer.

In one embodiment of the invention, an injectable nanoparticulate tacrolimus formulation is provided, comprising tacrolimus particles having an effective average particle size of less than about 600 nm and at least one surface stabilizer. In other embodiments, the injectable formulation can comprise tacrolimus having an effective average particle size of less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm. In one embodiment, the surface stabilizer is a povidone polymer.

The injectable nanoparticulate tacrolimus formulations of the invention eliminate the need to use polyoxyl 60 hydrogenated castor oil (HCO-60) as a solubilizer. This is beneficial, as in convention non-nanoparticulate injectable tacrolimus formulations comprising polyoxyl 60 hydrogenated castor oil as a solubilizer, the presence of this solubilizer can lead to anaphylactic shock (i.e., severe allergic reaction) and death. In addition, the injectable nanoparticulate tacrolimus formulations of the invention provide for formulations comprising high tacrolimus concentrations in low injection volumes, with rapid drug dissolution upon administration.

The present invention also describes pharmaceutical compositions comprising enteric-coated tacrolimus. Such formulations comprise nanoparticulate tacrolimus, having a particle size of less than about 2000 nm, and at least one surface stabilizer. The enteric coated dosage forms of the present invention may be provided in formulations which exhibit a variety of release profiles upon administration to a patient including, for example, an immediate-release (IR) formulation, a controlled-release (CR) formulation that allows once per day administration (or alternate time periods, such as once weekly or once monthly), and a combination of both IR and CR formulations. Because CR forms of the present invention can require only one dose per day, such dosage forms provide the benefits of enhanced patient convenience and compliance. The mechanism of controlled-release employed in the CR form may be accomplished in a variety of ways including, but not limited to, the use of erodable formulations, diffusion-controlled formulations, and osmotically-controlled formulations.

In another aspect of the invention there is provided a method of preparing the nanoparticulate tacrolimus formulations of the invention. The method comprises: (1) dispersing tacrolimus in a liquid dispersion medium; and (2) mechanically reducing the particle size of the tacrolimus to the desired effective average particle size, e.g., less than about 600 nm for injectable compositions or less than about 2000 nm for non-injectable or enteric-coated compositions. At least one surface stabilizer can be added to the dispersion media either before, during, or after particle size reduction of tacrolimus. In one embodiment for the injectable composition, the surface stabilizer is a povidone polymer with a molecular weight of less than about 40,000 daltons. Preferably, the liquid dispersion medium is maintained at a physiologic pH, for example, within the range of from about 3 to about 8, during the size reduction process.

The present invention is also directed to methods of treating a mammal, including a human, using the nanoparticulate tacrolimus formulations of the invention for the prophylaxis of organ rejection, and specifically in patients receiving allogenic liver or kidney transplants. Such methods comprise the step of administering to a subject a therapeutically effective amount of a nanoparticulate tacrolimus formulation of the invention, such as but not limited to an injectable or enteric-coated nanoparticulate tacrolimus formulation.

The nanoparticulate tacrolimus formulations of the present invention may optionally include one or more pharmaceutically acceptable excipients, such as non-toxic physiologically acceptable liquid carriers, pH adjusting agents, or preservatives.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1. Light micrograph using phase optics at 100× of unmilled tacrolimus.

FIG. 2. Light micrograph using phase optics at 100× of an aqueous dispersion of 10% (w/w) nanoparticulate tacrolimus (Camida LLC) with 2% (w/w) polyvinylpyrrolidone (PVP) K29/32 and 0.05% (w/w) dioctyl sulfosuccinate (DOSS).

FIG. 3: Light micrograph using phase optics at 100× of an aqueous dispersion of 10% (w/w) nanoparticulate tacrolimus (Camida LLC) with 2% (w/w) polyvinylpyrrolidone (PVP) K29/32 and 0.05% (w/w) dioctyl sulfosuccinate (DOSS) following one week of storage under refrigeration.

FIG. 4. Light micrograph using phase optics at 100× of an aqueous dispersion of 10% (w/w) nanoparticulate tacrolimus (Camida LLC), with 2% (w/w) PVP K12 and 0.15% (w/w) sodium deoxycholate.

FIG. 5. Light micrograph using phase optics at 100× of an aqueous dispersion of 20% (w/w) nanoparticulate tacrolimus (Camida LLC), with 3% (w/w) Plasdone® S630 (random copolymer of vinyl pyrrolidone and vinyl acetate in a 60:40 ratio).

FIG. 6. Light micrograph using phase optics at 100× of an aqueous dispersion of 20% (w/w) nanoparticulate tacrolimus (Camida LLC), with 3% (w/w) Plasdone® S630 (random copolymer of vinyl pyrrolidone and vinyl acetate in a 60:40 ratio) following one week of storage under refrigeration.

FIG. 7. Light micrograph using phase optics at 100× of an aqueous dispersion of 10% (w/w) nanoparticulate tacrolimus (Camida LLC), with 2% (w/w) hydroxypropylcellulose (HPC-SL) and 0.1% (w/w) DOSS.

FIG. 8. Light micrograph using phase optics at 100× of an aqueous dispersion of 5% (w/w) nanoparticulate tacrolimus (Camida LLC), with 1% (w/w) HPC-SL and 0.15% (w/w) DOSS.

FIG. 9. Light micrograph using phase optics at 100× of an aqueous dispersion of 5% (w/w) nanoparticulate tacrolimus (Camida LLC), with 1% (w/w) HPC-SL and 0.15% (w/w) DOSS following twelve days of storage under refrigeration.

FIG. 10. Light micrograph using phase optics at 100× of an aqueous dispersion of 5% (w/w) nanoparticulate tacrolimus (Camida LLC), with 1% (w/w) HPC-SL and 0.1% (w/w) sodium deoxycholate.

FIG. 11. Light micrograph using phase optics at 100× of an aqueous dispersion of 5% (w/w) nanoparticulate tacrolimus (Camida LLC), with 1% (w/w) HPC-SL and 0.1% (w/w) sodium deoxycholate following twelve days of storage under refrigeration.

FIG. 12. Light micrograph using phase optics at 100× of an aqueous dispersion of 10% (w/w) nanoparticulate tacrolimus (Camida LLC), with 2% (w/w) hydroxypropylmethyl cellulose (HPMC) and 0.05% (w/w) DOSS.

FIG. 13. Light micrograph using phase optics at 100× of an aqueous dispersion of 10% (w/w) nanoparticulate tacrolimus (Camida LLC), with 2% (w/w) hydroxypropylmethyl cellulose (HPMC) and 0.05% (w/w) DOSS following one week of storage under refrigeration.

FIG. 14. Light micrograph using phase optics at 100× of an aqueous dispersion of 10% (w/w) nanoparticulate tacrolimus (Camida LLC) with 2% Pluronic® F108.

FIG. 15. Light micrograph using phase optics at 100× of an aqueous dispersion of 10% (w/w) nanoparticulate tacrolimus (Camida LLC) with 2% Pluronic® F108 following one week of storage under refrigeration.

FIG. 16. Light micrograph using phase optics at 100× of an aqueous dispersion of 10% (w/w) nanoparticulate tacrolimus (Camida LLC) with 2% Tween® 80.

FIG. 17. Light micrograph using phase optics at 100× of an aqueous dispersion of 10% (w/w) nanoparticulate tacrolimus (Camida LLC) with 2% Tween® 80 following one week of storage under refrigeration.

### DETAILED DESCRIPTION OF THE INVENTION

### A. Introduction

The present invention is directed to compositions comprising a nanoparticulate formulation of tacrolimus and methods of making and using the same. The compositions comprise tacrolimus having an effective average particle size of less than about 2000 nm and at least one surface stabilizer.

Two examples of nanoparticulate tacrolimus dosage forms are an injectable nanoparticulate tacrolimus dosage form and an enteric coated nanoparticulate tacrolimus dosage form, although any pharmaceutically acceptable dosage form can be utilized. Examples of enteric coated dosage forms include, but are not limited to, solid dispersions or a liquid filled capsules of tacrolimus.

The dosage forms of the present invention may be provided in formulations which exhibit a variety of release profiles upon administration to a patient including, for example, an IR formulation, a CR formulation that allows once per day administration, and a combination of both IR and CR formulations. Because CR forms of the present invention can require only one dose per day (or one dose per suitable time period, such as weekly or monthly), such dosage forms provide the benefits of enhanced patient convenience and compliance. This is particularly beneficial for an immunosuppressant, as patient non-compliance with a dosage administration protocol can result in organ rejection. The mechanism of controlled-release employed in the CR form may be accomplished in a variety of ways including, but not limited to, the use of erodable formulations, diffusion-controlled formulations, and osmotically-controlled formulations.

The compositions described herein comprise nanoparticulate tacrolimus and at least one surface stabilizer. For the injectable compositions, the nanoparticulate tacrolimus preferably has an effective average particle size of less than about 600 nm. For the enteric coated compositions, the nanoparticulate tacrolimus has an effective average particle size of less than about 2000 nm.

Advantages of the nanoparticulate tacrolimus formulations of the present invention over conventional forms of tacrolimus (e.g., non-nanoparticulate or solubilized dosage forms) include, but are not limited to: (1) increased water solubility; (2) increased bioavailability; (3) smaller dosage form size due to enhanced bioavailability; (4) lower therapeutic dosages due to enhanced bioavailability; (5) reduced risk of unwanted side effects due to lower dosing; (6) enhanced patient convenience and compliance; and (7) more effective prophylaxis of organ rejection after organ replacement surgery. A further advantage of the injectable nanoparticulate tacrolimus formulation of the present invention over conventional forms of injectable tacrolimus is the elimination of the need to use polyoxyl 60 hydrogenated castor oil (HCO-60) as a solubilizer. A further advantage of the enteric coated nanoparticulate tacrolimus is a reduced risk of unwanted side effects due to the enteric coating.

The present invention also includes nanoparticulate tacrolimus compositions, together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracistemal, intraperitoneal, or topical administration, and the like.

### B. DEFINITIONS

The present invention is described herein using several definitions, as set forth below and throughout the application.

The term “effective average particle size of less than about 2000 nm”, as used herein means that at least 50% of the tacrolimus particles have a weight average size of less than about 2000 nm, when measured by, for example, sedimentation field flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to a stable tacrolimus particle connotes, but is not limited to one or more of the following parameters: (1), tacrolimus particles do not appreciably flocculate or agglomerate due to interparticle attractive forces or otherwise significantly increase in particle size over time; (2) that the physical structure of the tacrolimus particles is not altered over time, such as by conversion from an amorphous phase to a crystalline phase; (3) that the tacrolimus particles are chemically stable; and/or (4) where the tacrolimus has not been subject to a heating step at or above the melting point of the tacrolimus in the preparation of the nanoparticles of the present invention.

The term “conventional” or “non-nanoparticulate” active agent or tacrolimus shall mean an active agent, such as tacrolimus, which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, preferably less than about 20 mg/ml, preferably less than about 10 mg/ml, or preferably less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

The term “modified release” as used herein in relation to the composition according to the invention or a coating or coating material or used in any other context means release which is not immediate release and is taken to encompass controlled release, sustained release and delayed release.

The term “time delay” as used herein refers to the duration of time between administration of the composition and the release of tacrolimus from a particular component.

The term “lag time” as used herein refers to the time between delivery of active ingredient from one component and the subsequent delivery of tacrolimus from another component.

### C. Features of the Nanoparticulate Tacrolimus Compositions

There are a number of enhanced pharmacological characteristics of the nanoparticulate tacrolimus compositions of the present invention.

1. Increased Bioavailability

The tacrolimus formulations of the present invention exhibit increased bioavailability at the same dose of the same tacrolimus, and require smaller doses as compared to prior conventional tacrolimus formulations. Thus, a nanoparticulate tacrolimus tablet, if administered to a patient in a fasted state is not bioequivalent to administration of a conventional microcrystalline tacrolimus tablet in a fasted state.

The non-bioequivalence is significant because it means that the nanoparticulate tacrolimus dosage form exhibits significantly greater drug absorption. And for the nanoparticulate tacrolimus dosage form to be bioequivalent to the conventional microcrystalline tacrolimus dosage form, the nanoparticulate tacrolimus dosage form would have to contain significantly less drug. Thus, the nanoparticulate tacrolimus dosage form significantly increases the bioavailability of the drug.

Moreover, a nanoparticulate tacrolimus dosage form requires less drug to obtain the same pharmacological effect observed with a conventional microcrystalline tacrolimus dosage form (e.g., PROGRAF®). Therefore, the nanoparticulate tacrolimus dosage form has an increased bioavailability as compared to the conventional microcrystalline tacrolimus dosage form.

2. The Pharmacokinetic Profiles of the Tacrolimus Compositions of the Invention are not Affected by the Fed or Fasted State of the Subject Ingesting the Compositions

The compositions of the present invention encompass tacrolimus, wherein the pharmacokinetic profile of the tacrolimus is not substantially affected by the fed or fasted state of a subject ingesting the composition. This means that there is little or no appreciable difference in the quantity of drug absorbed or the rate of drug absorption when the nanoparticulate tacrolimus compositions are administered in the fed versus the fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance with tacrolimus, an increase in the medical condition for which the drug is being prescribed may be observed—i.e., the patient may suffer from organ rejection.

The invention also preferably provides tacrolimus compositions having a desirable pharmacokinetic profile when administered to mammalian subjects. The desirable pharmacokinetic profile of the tacrolimus compositions preferably includes, but is not limited to: (1) a Cₘₐₓ for tacrolimus, when assayed in the plasma of a mammalian subject following administration, that is preferably greater than the Cₘₐₓ for a non-nanoparticulate tacrolimus formulation (e.g., PROGRAF®), administered at the same dosage; and/or (2) an AUC for tacrolimus, when assayed in the plasma of a mammalian subject following administration, that is preferably greater than the AUC for a non-nanoparticulate tacrolimus formulation (e.g. PROGRAF®), administered at the same dosage; and/or (3) a Tmax for tacrolimus, when assayed in the plasma of a mammalian subject following administration, that is preferably less than the Tmax for a non-nanoparticulate tacrolimus formulation (e.g., PROGRAF®), administered at the same dosage. The desirable pharmacokinetic profile, as used herein, is the pharmacokinetic profile measured after the initial dose of tacrolimus.

In one embodiment, a preferred tacrolimus composition exhibits in comparative pharmacokinetic testing with a non-nanoparticulate tacrolimus formulation (e.g., PROGRAF®), administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate tacrolimus formulation.

In another embodiment, the tacrolimus composition of the invention exhibits in comparative pharmacokinetic testing with a non-nanoparticulate tacrolimus formulation of (e.g., PROGRAF®), administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate tacrolimus formulation.

In yet another embodiment, the tacrolimus composition of the invention exhibits in comparative pharmacokinetic testing with a non-nanoparticulate tacrolimus formulation (e.g., PROGRAF®), administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate tacrolimus formulation (e.g., PROGRAF®).

3. Bioequivalency of the Tacrolimus Compositions of the Invention when Administered in the Fed Versus the Fasted State

The invention also encompasses a composition comprising a nanoparticulate tacrolimus in which administration of the composition to a subject in a fasted state is bioequivalent to administration of the composition to a subject in a fed state.

The difference in absorption of the compositions comprising the nanoparticulate tacrolimus when administered in the fed versus the fasted state, is preferably less than about 35%, less than about 30%, less than about 25%, less than about 20%, less than about 15%, less than about 10%, less than about 5%, or less than about 3%.

In one embodiment of the invention, the invention encompasses nanoparticulate tacrolimus, wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

4. Dissolution Profiles of the Tacrolimus Compositions of the Invention

The tacrolimus compositions of the present invention have unexpectedly dramatic dissolution profiles. Rapid dissolution of an administered active agent is preferable, as faster dissolution generally leads to faster onset of action and greater bioavailability. To improve the dissolution profile and bioavailability of tacrolimus, it is useful to increase the drug's dissolution so that it could attain a level close to 100%.

The tacrolimus compositions of the present invention preferably have a dissolution profile in which within about 5 minutes at least about 20% of the composition is dissolved. In other embodiments of the invention, at least about 30% or about 40% of the tacrolimus composition is dissolved within about 5 minutes. In yet other embodiments of the invention, preferably at least about 40%, about 50%, about 60%, about 70%, or about 80% of the tacrolimus composition is dissolved within about 10 minutes. Finally, in another embodiment of the invention, preferably at least about 70%, about 80%, about 90%, or about 100% of the tacrolimus composition is dissolved within about 20 minutes.

Dissolution is preferably measured in a medium which is discriminating. Such a dissolution medium will produce two very different dissolution curves for two products having very different dissolution profiles in gastric juices, i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

5. Redispersibility Profiles of the Tacrolimus Compositions of the Invention

An additional feature of the tacrolimus compositions of the present invention is that the compositions redisperse such that the effective average particle size of the redispersed tacrolimus particles is less than about 2 microns. This is significant, as if upon administration the nanoparticulate tacrolimus compositions of the invention did not redisperse to a nanoparticulate particle size, then the dosage form may lose the benefits afforded by formulating the tacrolimus into a nanoparticulate particle size. A nanoparticulate size suitable for the present invention is an effective average particle size of less than about 2000 nm. In another embodiment, a nanoparticulate size suitable for the present invention is an effective average particle size of less than about 600 nm

Indeed, the nanoparticulate active agent compositions of the present invention benefit from the small particle size of the active agent; if the active agent does not redisperse into a small particle size upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall well below that observed with the liquid dispersion form of the nanoparticulate active agent.

Moreover, the nanoparticulate tacrolimus compositions of the invention exhibit dramatic redispersion of the nanoparticulate tacrolimus particles upon administration to a mammal, such as a human or animal, as demonstrated by reconstitution/redispersion in a biorelevant aqueous media such that the effective average particle size of the redispersed tacrolimus particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed tacrolimus particles of the invention (redispersed in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

6. Tacrolimus Compositions Used in Conjunction with Other Active Agents

The tacrolimus compositions of the invention can additionally comprise one or more compounds useful in the prophylaxis of organ rejection. The compositions of the invention can be co-formulated with such other active agents, or the compositions of the invention can be co-administered or sequentially administered in conjunction with such active agents. Examples of drugs that can be co-administered or co-formulated with tacrolimus include, but are not limited to, cyclosporine, mycophenolic acid, rapamycin (also known as sirolimus), alemtuzumab, mycophenolate mofetil, corticosteroids, glucocorticosteroids, doxycycline, interferon beta-1b, malononitrilamide FK778, azathioprine, Campath-1H, basiliximab, and methotrexate.

### D. Compositions

The invention provides compositions comprising nanoparticulate tacrolimus particles and at least one surface stabilizer. The surface stabilizers are preferably adsorbed to or associated with the surface of the tacrolimus particles. Surface stabilizers useful herein do not chemically react with the tacrolimus particles or itself. Preferably, individual molecules of the surface stabilizer are essentially free of intermolecular cross-linkages. In another embodiment, the compositions of the present invention can comprise two or more surface stabilizers.

The present invention also includes nanoparticulate tacrolimus compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracistemal, intraperitoneal, or topical administration, and the like. In certain embodiments of the invention, the nanoparticulate tacrolimus formulations are in an injectable form or an enteric coated oral form.

1. Tacrolimus

Tacrolimus, also known as FK-506 or Fujimycin, is a 23-membered macrolide lactone. As used herein, the term “tacrolimus” includes analogs and salts thereof, and can be in a crystalline phase, an amorphous phase, a semi-crystalline phase, a semi-amorphous phase, or a mixture thereof. The tacrolimus in the present invention, when applicable, may be present either in the form of one substantially optically pure enantiomer or as a mixture, racemic or otherwise, of enantiomers.

2. Surface Stabilizers

Combinations of more than one surface stabilizer can be used in the injectable tacrolimus formulation of the present invention. Suitable surface stabilizers include, but are not limited to, known organic and inorganic pharmaceutical excipients. Such excipients include various polymers, low molecular weight oligomers, natural products, and surfactants. Surface stabilizers include nonionic, anionic, cationic, ionic, and zwitterionic surfactants. A preferred surface stabilizer for an injectable nanoparticulate tacrolimus formulation is a povidone polymer.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween20® and Tween80® (ICI Speciality Chemicals)); polyethylene glycols (e.g., Carbowaxes 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-10G® or Surfactant 10-G® (Olin Chemicals, Stamford, Conn.); Crodestas SL-40® (Croda, Inc.); and SA9OHCO, which is C18H37CH2(CON(CH3)-CH2(CHOH)4(CH20H)2 (Eastman Kodak Co.); decanoyl-N-methylglucamide; n-decyl (-D-glucopyranoside; n-decyl (-D-maltopyranoside; n-dodecyl (-D-glucopyranoside; n-dodecyl (-D-maltoside; heptanoyl-N-methylglucamide; n-heptyl-(-D-glucopyranoside; n-heptyl (-D-thioglucoside; n-hexyl (-D-glucopyranoside; nonanoyl-N-methylglucamide; n-noyl (-D-glucopyranoside; octanoyl-N-methylglucamide; n-octyl-(-D-glucopyranoside; octyl (-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like. Also, if desirable, the nanoparticulate tacrolimus formulations of the present invention can be formulated to be phospholipid-free.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryl pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate. Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15-dimethyl hydroxyethyl ammonium chloride or bromide, coconut dimethyl hydroxyethyl ammonium chloride or bromide, myristyl trimethyl ammonium methyl sulfate, lauryl dimethyl benzyl ammonium chloride or bromide, lauryl dimethyl (ethenoxy)4 ammonium chloride or bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336), POLYQUAT, tetrabutylammonium bromide, benzyl trimethylammonium bromide, choline esters (such as choline esters of fatty acids), benzalkonium chloride, stearalkonium chloride compounds (such as stearyltrimonium chloride and distearyldimonium chloride), cetyl pyridinium bromide or chloride, halide salts of quaternized polyoxyethylalkylamines, MIRAPOL and ALKAQUAT (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants Physical Chemistry (Marcel Dekker, 1991); and J. Richmond, Cationic Surfactants Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quaternary phosphorous compound, a pyridinium compound, an anilinium compound, an ammonium compound, a hydroxylammonium compound, a primary ammonium compound, a secondary ammonium compound, a tertiary ammonium compound, and quaternary ammonium compounds of the formula NR1R2R3R4(+). For compounds of the formula NR1R2R3R4(+):

(i) none of R1-R4 are CH3;

(ii) one of R1-R4 is CH3;

(iii) three of R1-R4 are CH3;

(iv) all of R1-R4 are CH3;

(v) two of R1-R4 are CH3, one of R1-R4 is C6H5CH2, and one of R1-R4 is an alkyl chain of seven carbon atoms or less;

(vi) two of R1-R4 are CH3, one of R1-R4 is C6H5CH2, and one of R1-R4 is an alkyl chain of nineteen carbon atoms or more;

(vii) two of R1-R4 are CH3 and one of R1-R4 is the group C6H5(CH2)n, where n>1;

(viii) two of R1-R4 are CH3, one of R1-R4 is C6H5CH2, and one of R1-R4 comprises at least one heteroatom;

(ix) two of R1-R4 are CH3, one of R1-R4 is C6H5CH2, and one of R1-R4 comprises at least one halogen;

(x) two of R1-R4 are CH3, one of R1-R4 is C6H5CH2, and one of R1-R4 comprises at least one cyclic fragment;

(xi) two of R1-R4 are CH3 and one of R1-R4 is a phenyl ring; or

(xii) two of R1-R4 are CH3 and two of R1-R4 are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquarternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated herein by reference.

Povidone Polymers

Povidone polymers are preferred surface stabilizers for use in formulating an injectable nanoparticulate tacrolimus formulation. Povidone polymers, also known as polyvidon(e), povidonum, PVP, and polyvinylpyrrolidone, are sold under the trade names Kollidon® (BASF Corp.) and Plasdone® (ISP Technologies, Inc.). They are polydisperse macromolecular molecules, with a chemical name of 1-ethenyl-2-pyrrolidinone polymers and 1-vinyl-2-pyrrolidinone polymers. Povidone polymers are produced commercially as a series of products having mean molecular weights ranging from about 10,000 to about 700,000 daltons. To be useful as a surface modifier for a drug compound to be administered to a mammal, the povidone polymer must have a molecular weight of less than about 40,000 daltons, as a molecular weight of greater than 40,000 daltons would have difficulty clearing the body.

Povidone polymers are prepared by, for example, Reppe's process, comprising: (1) obtaining 1,4-butanediol from acetylene and formaldehyde by the Reppe butadiene synthesis; (2) dehydrogenating the 1,4-butanediol over copper at 200° to form γ-butyrolactone; and (3) reacting γ-butyrolactone with ammonia to yield pyrrolidone. Subsequent treatment with acetylene gives the vinyl pyrrolidone monomer. Polymerization is carried out by heating in the presence of H₂O and NH₃. See The Merck Index, 10^{th} Edition, pp. 7581 (Merck & Co., Rahway, N.J., 1983).

The manufacturing process for povidone polymers produces polymers containing molecules of unequal chain length, and thus different molecular weights. The molecular weights of the molecules vary about a mean or average for each particular commercially available grade. Because it is difficult to determine the polymer's molecular weight directly, the most widely used method of classifying various molecular weight grades is by K-values, based on viscosity measurements. The K-values of various grades of povidone polymers represent a function of the average molecular weight, and are derived from viscosity measurements and calculated according to Fikentscher's formula.

The weight-average of the molecular weight, Mw, is determined by methods that measure the weights of the individual molecules, such as by light scattering. Table 1 provides molecular weight data for several commercially available povidone polymers, all of which are soluble.

TABLE 1

| Povidone | K-Value | Mv (Daltons)** | Mw (Daltons)** | Mn (Daltons)** |
|---|---|---|---|---|
| Plasdone C-15 ® | 17 ± 1 | 7,000 | 10,500 | 3,000 |
| Plasdone C-30 ® | 30.5 ± 1.5 | 38,000 | 62,500* | 16,500 |
| Kollidon 12 PF ® | 11-14 | 3,900 | 2,000-3,000 | 1,300 |
| Kollidon 17 PF ® | 16-18 | 9,300 | 7,000-11,000 | 2,500 |
| Kollidon 25 ® | 24-32 | 25,700 | 28,000-34,000 | 6,000 |

*Because the molecular weight is greater than 40,000 daltons, this povidone polymer is not useful as a surface stabilizer for a drug compound to be administered parenterally (i.e., injected).
**Mv is the viscosity-average molecular weight, Mn is the number-average molecular weight, and Mw is the weight average molecular weight. Mw and Mn were determined by light scattering and ultra-centrifugation, and Mv was determined by viscosity measurements.

Based on the data provided in Table 1, exemplary preferred commercially available povidone polymers include, but are not limited to, Plasdone C-15®, Kollidon 12 PF®, Kollidon 17 PF®, and Kollidon 25®.

3. Nanoparticulate Tacrolimus Particle Size

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

Compositions of the invention, and the enteric coated compositions in particular, comprise tacrolimus nanoparticles having an effective average particle size of less than about 2000 nm (i.e., 2 microns). In other embodiments of the invention, the tacrolimus nanoparticles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

In another embodiment, the nanoparticulate compositions of the present invention, and the injectable nanoparticulate compositions in particular, comprise tacrolimus nanoparticles that have an effective average particles size of less than about 600 nm. In other embodiments, the effective average particle size is less than about 550 nm, less than about 500 nm, less than about 450 nm, less than about 400 nm, less than about 300 nm, less than about 250 nm, less than about 200 nm, less than about 150 nm, less than about 100 nm, less than about 75 nm, or less than about 50 nm.

An “effective average particle size of less than about 2000 nm” means that at least 50% of the tacrolimus particles have a particle size less than the effective average, by weight, i.e., less than about 2000 nm. If the “effective average particle size” is less than about 1900 nm, then at least about 50% of the tacrolimus particles have a size of less than about 1900 nm, when measured by the above-noted techniques. The same is true for the other particle sizes referenced above. In other embodiments, at least about 70%, at least about 90%, at least about 95%, or at least about 99% of the tacrolimus particles have a particle size less than the effective average, i.e., less than about 2000 nm, about 1900 nm, about 1800 nm, etc.

In the present invention, the value for D50 of a nanoparticulate tacrolimus composition is the particle size below which 50% of the tacrolimus particles fall, by weight. Similarly, D90 is the particle size below which 90% of the tacrolimus particles fall, by weight.

4. Concentration of Nanoparticulate Tacrolimus and Surface Stabilizers

The relative amounts of tacrolimus and one or more surface stabilizers can vary widely. The optimal amount of the individual components depends, for example, upon physical and chemical attributes of the surface stabilizer(s) selected, such as the hydrophilic lipophilic balance (HLB), melting point, and the surface tension of water solutions of the stabilizer, etc.

Preferably, the concentration of tacrolimus can vary from about 99.5% to about 0.001%, from about 95% to about 0.1%, or from about 90% to about 0.5%, by weight, based on the total combined weight of the tacrolimus and at least one surface stabilizer, not including other excipients. Higher concentrations of the active ingredient are generally preferred from a dose and cost efficiency standpoint.

Preferably, the concentration of surface stabilizer can vary from about 0.5% to about 99.999%, from about 5.0% to about 99.9%, or from about 10% to about 99.5%, by weight, based on the total combined dry weight of tacrolimus and at least one surface stabilizer, not including other excipients.

5. Other Pharmaceutical Excipients

Pharmaceutical compositions of the invention may also comprise one or more binding agents, filling agents, lubricating agents, suspending agents, sweeteners, flavoring agents, preservatives, buffers, wetting agents, disintegrants, effervescent agents, and other excipients depending upon the route of administration and the dosage form desired. Such excipients are well known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC®).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, and quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples, such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

6. Injectable Nanoparticulate Tacrolimus Formulations

The invention provides injectable nanoparticulate tacrolimus formulations that can comprise high drug concentrations in low injection volumes, with rapid drug dissolution upon administration. In addition, the injectable nanoparticulate tacrolimus formulation of the invention eliminate the need to use polyoxyl 60 hydrogenated castor oil (HCO-60) as a solubilizer.

An exemplary injectable tacrolimus formulation comprises, based on % w/w:

| | |
|---|---|
| Tacrolimus | 5-50% |
| povidone polymer | 0.1-50% |
| preservatives | 0.05-0.25% |
| pH adjusting agent | pH about 6 to about 7 |
| water for injection | q.s. |

Exemplary preservatives include methylparaben (about 0.18% based on % w/w), propylparaben (about 0.02% based on % w/w), phenol (about 0.5% based on % w/w), and benzyl alcohol (up to 2% v/v). An exemplary pH adjusting agent is sodium hydroxide, and an exemplary liquid carrier is sterile water for injection. Other useful preservatives, pH adjusting agents, and liquid carriers are well-known in the art.

The tacrolimus is preferably present in an injectable nanoparticulate formulation of the present invention in an amount of from about 0.01 mg to about 50 mg, preferably in the amount of from about 0.05 mg to about 20 mg.

7. Enteric Coated Oral Formulations

Tacrolimus bioavailability is reduced when administered with food. Administration with food causes an increase in the amount of time that the tacrolimus is retained in the stomach. This increased retention time allows the tacrolimus to dissolve in the acidic stomach conditions. Then, when the dissolved drug exits the stomach and enters the more basic conditions of the upper small intestine, the tacrolimus precipitates out of solution. The precipitated tacrolimus is poorly absorbed since it must once again dissolve before it can be absorbed and this process is slow because of the poor water solubility of tacrolimus. The dissolving of the drug in the stomach, followed by precipitation, diminishes the enhanced bioavailability that tacrolimus can gain from administration as a nanoparticulate dosage form, such as a nanoparticulate tacrolimus solid dispersion, or nanoparticulate tacrolimus liquid filled capsule. Protection of the drug from the low pH conditions of the stomach would reduce or eliminate this decrease in bioavailability. In addition, an enteric coating would decrease or eliminate the nausea and vomiting associate with tacrolimus administration.

Therefore, a composition comprising enteric-coated nanoparticulate tacrolimus is described herein. In one embodiment, the oral formulation comprises an enteric coated solid dosage form.

Solid dosage forms for oral administration include, but are not limited to, capsules, tablets, pills, powders, and granules. In such solid dosage forms, the tacrolimus is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Drug Release Profiles

In one embodiment, the enteric-coated tacrolimus composition described herein exhibits a pulsatile plasma profile when administered to a patient in an oral dosage form. The plasma profile associated with the administration of a drug compound may be described as a “pulsatile profile” in which pulses of high tacrolimus concentration, interspersed with low concentration troughs, are observed. A pulsatile profile containing two peaks may be described as “bimodal”. Similarly, a composition or a dosage form which produces such a profile upon administration may be said to exhibit “pulsed release” of tacrolimus.

Conventional frequent dosage regimes in which an immediate release (IR) dosage form is administered at periodic intervals typically gives rise to a pulsatile plasma profile. In this case, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash out period provided by the fall off of the plasma concentration of tacrolimus between peaks has been thought to be a contributing factor in reducing or preventing patient tolerance to various types of drugs.

Multiparticulate modified controlled release (CR) compositions similar to those disclosed herein are disclosed and claimed in the U.S. Pat. Nos. 6,228,398, 6,730,325 and 6,793,936 to Devane et al; all of which are specifically incorporated by reference herein. All of the relevant prior art in this field may be found therein.

Another aspect of the present invention is a multiparticulate modified release composition having a first component comprising a first population of tacrolimus and a second component comprising a second population of tacrolimus. The ingredient-containing particles of the second component are coated with a modified release coating. Alternatively or additionally, the second population of tacrolimus-containing particles further comprises a modified release matrix material. Following oral delivery, the composition in operation delivers the tacrolimus in a pulsatile manner.

In a preferred embodiment of a multiparticulate modified release composition according to the invention, the first component is an immediate release component.

The modified release coating applied to the second population of tacrolimus particles causes a lag time between the release of active from the first population of tacrolimus-containing particles and the release of active from the second population of active tacrolimus-containing particles. Similarly, the presence of a modified release matrix material in the second population of tacrolimus-containing particles causes a lag time between the release of tacrolimus from the first population of tacrolimus-containing particles and the release of active ingredient from the second population of tacrolimus-containing particles. The duration of the lag time may be varied by altering the composition and/or the amount of the modified release coating and/or altering the composition and/or amount of modified release matrix material utilized. Thus, the duration of the lag time can be designed to mimic a desired plasma profile.

Because the plasma profile produced by the multiparticulate modified release composition upon administration is substantially similar to the plasma profile produced by the administration of two or more IR dosage forms given sequentially, the multiparticulate controlled release composition of the present invention is particularly useful for administering tacrolimus for which patient tolerance may be problematical. This multiparticulate modified release composition is therefore advantageous for reducing or minimizing the development of patient tolerance to the active ingredient in the composition.

The present invention further provides a method for prophylaxis of organ rejection comprising administering a therapeutically effective amount of a composition or solid oral dosage form according to the present invention to provide pulsed or bimodal administration of tacrolimus. Advantages of the present invention include reducing the dosing frequency required by conventional multiple IR dosage regimes while still maintaining the benefits derived from a pulsatile plasma profile. This reduced dosing frequency is advantageous in terms of patient compliance to have a formulation which may be administered at reduced frequency. The reduction in dosage frequency made possible by utilizing the present invention would contribute to reducing health care costs by reducing the amount of time spent by health care workers on the administration of drugs.

The active ingredient in each component may be the same or different. For example, a composition in which the first component contains tacrolimus and the second component comprises a second active ingredient may be desirable for combination therapies. Indeed, two or more active ingredients may be incorporated into the same component when the active ingredients are compatible with each other. A drug compound present in one component of the composition may be accompanied by, for example, an enhancer compound or a sensitizer compound in another component of the composition, to modify the bioavailability or therapeutic effect of the drug compound.

As used herein, the term “enhancer” refers to a compound which is capable of enhancing the absorption and/or bioavailability of an active ingredient by promoting net transport across the GIT in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

The proportion of tacrolimus contained in each component may be the same or different depending on the desired dosing regime. The tacrolimus is present in the first component and in the second component in any amount sufficient to elicit a therapeutic response. The tacrolimus when applicable, may be present either in the form of one substantially optically pure enantiomer or as a mixture, racemic or otherwise, of enantiomers. The tacrolimus is preferably present in a composition in an amount of from 0.1-60 mg, preferably in the amount of from 1-30 mg. Tacrolimus is preferably present in the first component in an amount of from 0.5-60 mg; more preferably the tacrolimus is present in the first component in an amount of from 2.5-30 mg. The tacrolimus is present in the subsequent components in an amount within a similar range to that described for the first component.

The time-release characteristics for the release of tacrolimus from each of the components may be varied by modifying the composition of each component, including modifying any of the excipients or coatings which may be present. In particular the release of tacrolimus may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time or delay time for the release of tacrolimus from each component may also be varied by modifying the composition of each of the components, including modifying any excipients and coatings which may be present. For example, the first component may be an immediate release component wherein the tacrolimus is released substantially immediately upon administration. Alternatively, the first component may be, for example, a time-delayed immediate release component in which the tacrolimus is released substantially immediately after a time delay. The second component may be, for example, a time-delayed immediate release component as just described or, alternatively, a time-delayed sustained release or extended release component in which the tacrolimus is released in a controlled fashion over an extended period of time.

As will be appreciated by those skilled in the art, the exact nature of the plasma concentration curve will be influenced by the combination of all of these factors just described. In particular, the lag time between the delivery (and thus also the onset of action) of the tacrolimus in each component may be controlled by varying the composition and coating (if present) of each of the components. Thus by variation of the composition of each component (including the amount and nature of the active ingredient(s)) and by variation of the lag time, numerous release and plasma profiles may be obtained. Depending on the duration of the lag time between the release of tacrolimus from each component and the nature of the release from each component (i.e. immediate release, sustained release etc.), the pulses in the plasma profile may be well separated and clearly defined peaks (e.g. when the lag time is long) or the pulses may be superimposed to a degree (e.g. in when the lag time is short).

In a preferred embodiment, the multiparticulate modified release composition according to the present invention has an immediate release component and at least one modified release component, the immediate release component comprising a first population of tacrolimus-containing particles and the modified release components comprising second and subsequent populations of tacrolimus-containing particles. The second and subsequent modified release components may comprise a controlled release coating. Additionally or alternatively, the second and subsequent modified release components may comprise a modified release matrix material. In operation, administration of such a multiparticulate modified release composition having, for example, a single modified release component results in characteristic pulsatile plasma concentration levels of the tacrolimus in which the immediate release component of the composition gives rise to a first peak in the plasma profile and the modified release component gives rise to a second peak in the plasma profile. Embodiments of the invention comprising more than one modified release component give rise to further peaks in the plasma profile.

Such a plasma profile produced from the administration of a single dosage unit is advantageous when it is desirable to deliver two (or more) pulses of tacrolimus without the need for administration of two (or more) dosage units.

Enteric Coating

Any coating material which modifies the release of the tacrolimus in the desired manner may be used. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit® RS and RL, poly acrylic acid and poly acrylate and methacrylate copolymers such as those sold under the Trade Mark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. about 5 k-5,000 k), polyvinylpyrrolidone (m. wt. about 10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. about 30 k-300 k), polysaccharides such as agar, acacia, karaya, tragacanth, algins and guar, polyacrylamides, Polyox polyethylene oxides (m. wt. about 100 k-5,000 k), AquaKeep acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticizers, lubricants, solvents and the like may be added to the coating. Suitable plasticizers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropionin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of tacrolimus dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

A multiparticulate modified release composition according to the present invention may be incorporated into any suitable dosage form which facilitates release of the active ingredient in a pulsatile manner. Typically, the dosage form may be a blend of the different populations of tacrolimus-containing particles which make up the immediate release and the modified release components, the blend being filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multi-layer tablet. In this instance the first component of the multiparticulate modified release composition may be compressed into one layer, with the second component being subsequently added as a second layer of the multi-layer tablet. The populations of tacrolimus-containing particles making up the composition of the invention may further be included in rapidly dissolving dosage forms such as an effervescent dosage form or a fast-melt dosage form.

In another embodiment, the composition according to the invention comprises at least two populations of tacrolimus-containing particles which have different in vitro dissolution profiles.

Preferably, in operation the composition of the invention and the solid oral dosage forms containing the composition release the tacrolimus such that substantially all of the tacrolimus contained in the first component is released prior to release of the tacrolimus from the second component. When the first component comprises an IR component, for example, it is preferable that release of the tacrolimus from the second component is delayed until substantially all the tacrolimus in the IR component has been released. Release of the tacrolimus from the second component may be delayed as detailed above by the use of a modified release coating and/or a modified release matrix material.

In one embodiment, when it is desirable to minimize patient tolerance by providing a dosage regime which facilitates wash-out of a first dose of tacrolimus from a patient's system, release of the tacrolimus from the second component is delayed until substantially all of the tacrolimus contained in the first component has been released, and further delayed until at least a portion of the tacrolimus released from the first component has been cleared from the patient's system. In a particular embodiment, release of the tacrolimus from the second component of the composition in operation is substantially, if not completely, delayed for a period of at least about two hours after administration of the composition.

The release of the drug from the second component of the composition in operation is substantially, if not completely, delayed for a period of at least about four hours, preferably about four hours, after administration of the composition.

### E. Methods of Making Nanoparticulate Tacrolimus Formulations

Nanoparticulate tacrolimus compositions can be made using any suitable method known in the art such as, for example, milling, homogenization, or precipitation techniques. Exemplary methods of making nanoparticulate compositions are described in U.S. Pat. No. 5,145,684. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated herein by reference.

The resultant nanoparticulate tacrolimus compositions or dispersions can be utilized in solid, semi-solid, or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

Consistent with the above disclosure, provided herein is a method of preparing the nanoparticulate tacrolimus formulations of the invention. The method comprises the steps of: (1) dispersing tacrolimus in a liquid dispersion medium; and (2) mechanically reducing the particle size of the tacrolimus to the desired effective average particle size, such as less than about 2000 nm or less than about 600 nm. A surface stabilizer can be added before, during, or after particle size reduction of tacrolimus. The liquid dispersion medium can be maintained at a physiologic pH, for example, within the range of from about 3.0 to about 8.0 during the size reduction process; more preferably within the range of from about 5.0 to about 7.5 during the size reduction process. The dispersion medium used for the size reduction process is preferably aqueous, although any media in which tacrolimus is poorly soluble and dispersible can be used, such as safflower oil, ethanol, t-butanol, glycerin, polyethylene glycol (PEG), hexane, or glycol.

Effective methods of providing mechanical force for particle size reduction of tacrolimus include ball milling, media milling, and homogenization, for example, with a Microfluidizer® (Microfluidics Corp.). Ball milling is a low energy milling process that uses milling media, drug, stabilizer, and liquid. The materials are placed in a milling vessel that is rotated at optimal speed such that the media cascades and reduces the drug particle size by impaction. The media used must have a high density as the energy for the particle reduction is provided by gravity and the mass of the attrition media.

Media milling is a high energy milling process. Drug, stabilizer, and liquid are placed in a reservoir and recirculated in a chamber containing media and a rotating shaft/impeller. The rotating shaft agitates the media which subjects the drug to impaction and sheer forces, thereby reducing the drug particle size.

Homogenization is a technique that does not use milling media. Drug, stabilizer, and liquid (or drug and liquid with the stabilizer added after particle size reduction) constitute a process stream propelled into a process zone, which in the Microfluidizer® is called the Interaction Chamber. The product to be treated is inducted into the pump, and then forced out. The priming valve of the Microfluidizer® purges air out of the pump. Once the pump is filled with product, the priming valve is closed and the product is forced through the interaction chamber. The geometry of the interaction chamber produces powerful forces of sheer, impact, and cavitation which are responsible for particle size reduction. Specifically, inside the interaction chamber, the pressurized product is split into two streams and accelerated to extremely high velocities. The formed jets are then directed toward each other and collide in the interaction zone. The resulting product has very fine and uniform particle or droplet size. The Microfluidizer® also provides a heat exchanger to allow cooling of the product. U.S. Pat. No. 5,510,118, which is specifically incorporated by reference, refers to a process using a Microfluidizer®.

Using a particle size reduction method, the particle size of tacrolimus is reduced to the desired an effective average particle size, such as less than about 2000 nm for the enteric coated formulation, and less than about 600 nm for the injectable tacrolimus formulation.

Tacrolimus can be added to a liquid medium in which it is essentially insoluble to form a premix. The concentration of the tacrolimus in the liquid medium can vary from about 5 to about 60%, and preferably is from about 15 to about 50% (w/v), and more preferably about 20 to about 40%. The surface stabilizer can be present in the premix or it can be added to the drug dispersion following particle size reduction. The concentration of the surface stabilizer can vary from about 0.1 to about 50%, and preferably is from about 0.5 to about 20%, and more preferably from about 1 to about 10%, by weight.

The premix can be used directly by subjecting it to mechanical means to reduce the average tacrolimus particle size in the dispersion to less than about 600 nm. It is preferred that the premix be used directly when a ball mill is used for attrition. Alternatively, tacrolimus and at least one surface stabilizer can be dispersed in the liquid medium using suitable agitation, e.g., a Cowles type mixer, until a homogeneous dispersion is observed in which there are no large agglomerates visible to the naked eye. It is preferred that the premix be subjected to such a premilling dispersion step when a recirculating media mill is used for attrition.

The mechanical means applied to reduce the tacrolimus particle size conveniently can take the form of a dispersion mill. Suitable dispersion mills include a ball mill, an attritor mill, a vibratory mill, and media mills such as a sand mill and a bead mill. A media mill is preferred due to the relatively shorter milling time required to provide the desired reduction in particle size. For media milling, the apparent viscosity of the premix is preferably from about 100 to about 1000 centipoise, and for ball milling the apparent viscosity of the premix is preferably from about 1 up to about 100 centipoise. Such ranges tend to afford an optimal balance between efficient particle size reduction and media erosion.

The attrition time can vary widely and depends primarily upon the particular mechanical means and processing conditions selected. For ball mills, processing times of up to five days or longer may be required. Alternatively, processing times of less than 1 day (residence times of one minute up to several hours) are possible with the use of a high shear media mill.

The tacrolimus particles must be reduced in size at a temperature which does not significantly degrade tacrolimus. Processing temperatures of less than about 30 to less than about 40° C. are ordinarily preferred. If desired, the processing equipment can be cooled with conventional cooling equipment. Control of the temperature, e.g., by jacketing or immersion of the milling chamber in ice water, is contemplated. Generally, the method of the invention is conveniently carried out under conditions of ambient temperature and at processing pressures which are safe and effective for the milling process. Ambient processing pressures are typical of ball mills, attritor mills, and vibratory mills.

Grinding Media

The grinding media can comprise particles that are preferably substantially spherical in shape, e.g., beads, consisting essentially of polymeric resin. Alternatively, the grinding media can comprise a core having a coating of a polymeric resin adhered thereon.

In general, suitable polymeric resins are chemically and physically inert, substantially free of metals, solvent, and monomers, and of sufficient hardness and friability to enable them to avoid being chipped or crushed during grinding. Suitable polymeric resins include crosslinked polystyrenes, such as polystyrene crosslinked with divinylbenzene; styrene copolymers; polycarbonates; polyacetals, such as Delrin® (E.I. du Pont de Nemours and Co.); vinyl chloride polymers and copolymers; polyurethanes; polyamides; poly(tetrafluoroethylenes), e.g., Teflon® (E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes and the like. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 0.01 to about 3 mm. For fine grinding, the grinding media is preferably from about 0.02 to about 2 mm, and more preferably from about 0.03 to about 1 mm in size.

The polymeric resin can have a density from about 0.8 to about 3.0 g/cm³.

In a preferred grinding process the particles are made continuously. Such a method comprises continuously introducing tacrolimus into a milling chamber, contacting the tacrolimus with grinding media while in the chamber to reduce the tacrolimus particle size, and continuously removing the nanoparticulate tacrolimus from the milling chamber.

The grinding media is separated from the milled nanoparticulate tacrolimus using conventional separation techniques, in a secondary process such as by simple filtration, sieving through a mesh filter or screen, and the like. Other separation techniques such as centrifugation may also be employed.

Sterile Product Manufacturing

Development of injectable compositions requires the production of a sterile product. The manufacturing process of the present invention is similar to typical known manufacturing processes for sterile suspensions. A typical sterile suspension manufacturing process flowchart is as follows:

[symbol]

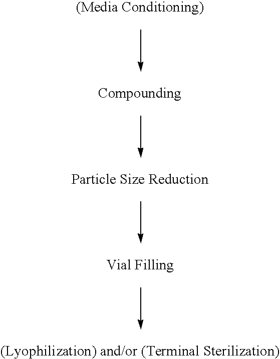

As indicated by the optional steps in parentheses, some of the processing is dependent upon the method of particle size reduction and/or method of sterilization. For example, media conditioning is not required for a milling method that does not use media. If terminal sterilization is not feasible due to chemical and/or physical instability, aseptic processing can be used.

### F. Methods of Treatment

In human therapy, it is important to provide a tacrolimus dosage form that delivers the required therapeutic amount of the drug in vivo, and that renders the drug bioavailable in a constant manner. Thus, another aspect of the present invention provides a method of treating a mammal, including a human, using a nanoparticulate tacrolimus formulation of the invention for the prophylaxis of organ rejection, and specifically in patients receiving allogenic liver or kidney transplants. Such methods comprise the step of administering to a subject a therapeutically effective amount of a nanoparticulate tacrolimus formulation of the present invention. In one embodiment, the nanoparticulate tacrolimus formulation is an injectable formulation. In another embodiment, the nanoparticulate tacrolimus formulation is an enteric coated oral formulation.

One of ordinary skill will appreciate that effective amounts of a tacrolimus can be determined empirically and can be employed in pure form or, where such forms exist, in pharmaceutically acceptable salt, ester, or prod rug form. Actual dosage levels of tacrolimus in the enteric-coated compositions of the invention may be varied to obtain an amount of tacrolimus that is effective to obtain a desired therapeutic response for a particular composition and method of administration. The selected dosage level therefore depends upon the desired therapeutic effect, the route of administration, the potency of the administered tacrolimus, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following examples are given to illustrate the present invention. It should be understood, however, that the spirit and scope of the invention is not to be limited to the specific conditions or details described in these examples but should only be limited by the scope of the claims that follow. All references identified herein, including U.S. patents, are hereby expressly incorporated by reference.

The following examples are given to illustrate the present invention. It should be understood, however, that the spirit and scope of the invention is not to be limited to the specific conditions or details described in these examples but should only be limited by the scope of the claims that follow. All references identified herein, including U.S. patents, are hereby expressly incorporated by reference.

### EXAMPLES

### Example 1

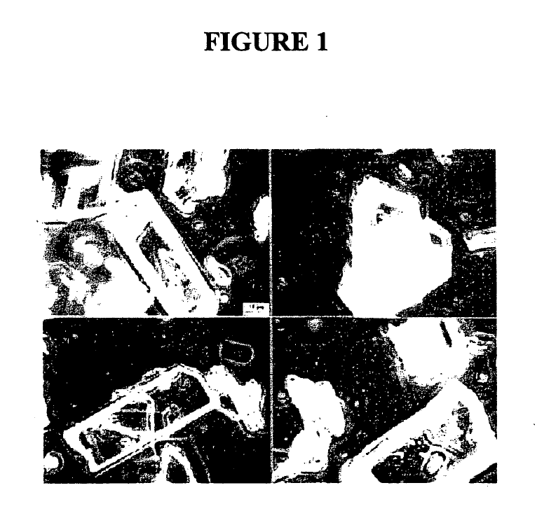

The purpose of this example was to prepare a nanoparticulate tacrolimus formulation. FIG. 1 shows a light micrograph using phase optics at 100× of unmilled tacrolimus.

An aqueous dispersion of 10% (w/w) tacrolimus (Camida LLC), combined with 2% (w/w) polyvinylpyrrolidone (PVP) K29/32 and 0.05% (w/w) dioctylsulfosuccinate (DOSS), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes.

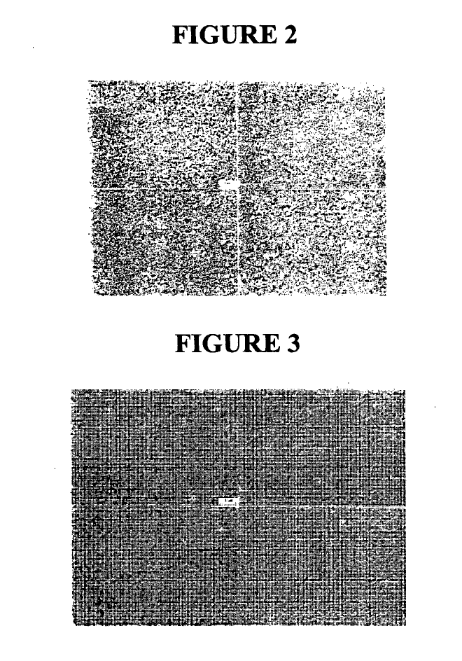

Following milling, the particle size of the milled tacrolimus particles was measured, in deionized distilled water, using a Horiba LA 910 particle size analyzer. The initial mean milled tacrolimus particle size was 192 nm, with a D50 of 177 nm and a D90 of 278 nm. FIG. 2 shows a light micrograph using phase optics at 100× of the milled tacrolimus. In a second measurement in distilled water following 1 week of refrigeration at <15° C., the mean tacrolimus particle size was 245 nm, with a D50 of 219 nm and a D90 of 374 mm. FIG. 3 shows a light micrograph using phase optics at 100× of the milled tacrolimus following one week of refrigeration.

The results demonstrate the successful preparation of a stable nanoparticulate tacrolimus formulation, as the mean particle size obtained was 192 nm, and minimal particle size growth was observed following storage.

### Example 2

The purpose of this example was to prepare a nanoparticulate tacrolimus formulation.

An aqueous dispersion of 10% (w/w) tacrolimus (Camida LLC), combined with 2% PVP K12 and 0.15% sodium deoxycholate, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 150 minutes.

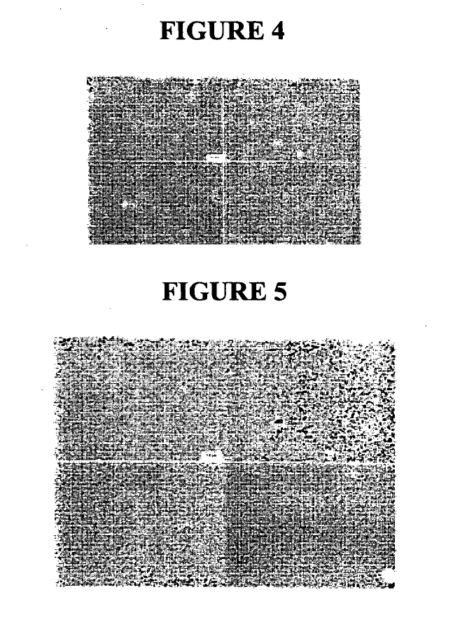

Following milling, the particle size of the milled tacrolimus particles was measured, in deionized distilled water, using a Horiba LA 910 particle size analyzer. The mean milled tacrolimus particle size was 329 nm, with a D50 of 303 nm and a D90 of 466 nm. FIG. 4 shows a light micrograph using phase optics at 100× of the milled tacrolimus.

The results demonstrate the successful preparation of a stable nanoparticulate tacrolimus formulation, as the mean particle size obtained was 329 nm.

### Example 3

The purpose of this example was to prepare a nanoparticulate tacrolimus formulation.

An aqueous dispersion of 20% (w/w) tacrolimus (Camida LLC), combined with 3% (w/w) Pluronic® S630 and 0.05% (w/w) DOSS, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes. A light micrograph using phase optics at 100× of the milled tacrolimus is shown in FIG. 5.

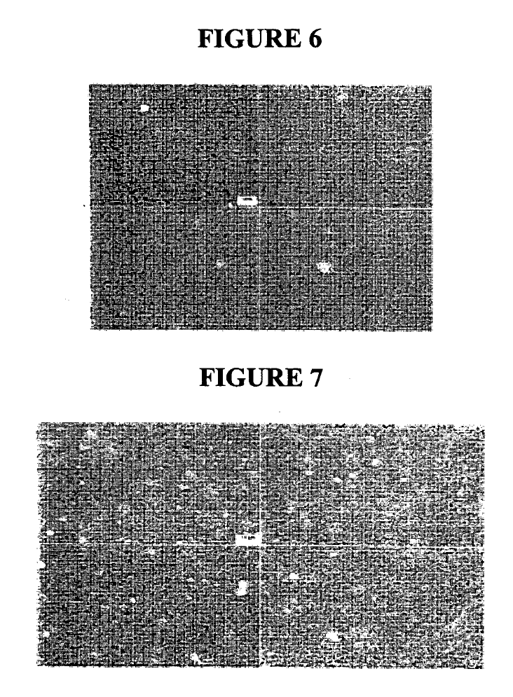

Following milling, the particle size of the milled tacrolimus particles was measured, in deionized distilled water, using a Horiba LA 910 particle size analyzer. The initial mean milled tacrolimus particle size was 171 nm, with a D50 of 163 nm and a D90 of 230 nm. In a second measurement in distilled water following 1 week of refrigeration at <15° C., the mean tacrolimus particle size was 194 nm, with a D50 of 180 nm and a D90 of 279 nm. A light micrograph using phase optics at 100× of the milled tacrolimus following one week of storage under refrigeration is shown in FIG. 6.

The results demonstrate the successful preparation of a stable nanoparticulate tacrolimus formulation, as the mean particle size obtained was 171 nm, and minimal particle size growth was observed following storage.

### Example 4

The purpose of this example was to prepare a nanoparticulate tacrolimus formulation.

An aqueous dispersion of 10% (w/w) tacrolimus (Camida LLC), combined with 2% (w/w) hydroxypropylcellulose (HPC-SL) and 0.1% (w/w) DOSS, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 150 minutes. A light micrograph using phase optics at 100× of the milled tacrolimus is shown in FIG. 7.

Following milling, the particle size of the milled tacrolimus particles was measured, in deionized distilled water, using a Horiba LA 910 particle size analyzer. The mean milled tacrolimus particle size was 389 nm, with a D50 of 328 nm and a D90 of 614 nm.

The results demonstrate the successful preparation of a stable nanoparticulate tacrolimus formulation, as the mean particle size obtained was 389 nm.

### Example 5

The purpose of this example was to prepare a nanoparticulate tacrolimus formulation.

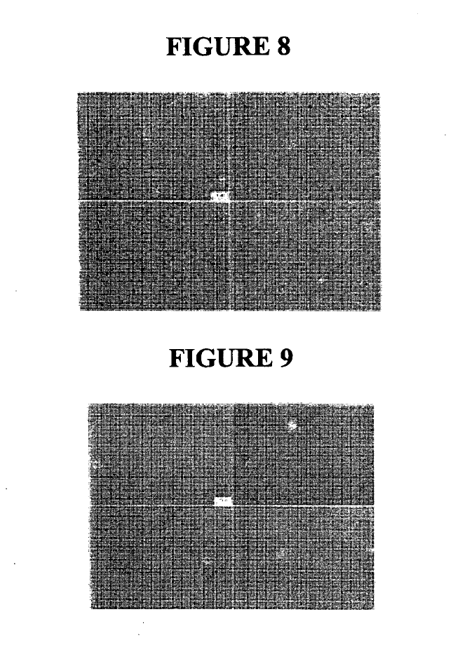

An aqueous dispersion of 5% (w/w) tacrolimus (Camida LLC), combined with 1% (w/w) HPC-SL and 0.15% (w/w) DOSS, was milled in a 10 ml chamber of a NanoMillg 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 5500 rpms for 90 minutes. A light micrograph using phase optics at 100× of the milled tacrolimus is shown in FIG. 8.

Following milling, the particle size of the milled tacrolimus particles was measured, in deionized distilled water, using a Horiba LA 910 particle size analyzer. The initial mean milled tacrolimus particle size was 169 nm, with a D50 of 160 nm and a D90 of 225 nm. In a second measurement in distilled water following 12 days of refrigeration at <15° C., the mean tacrolimus particle size was 155 nm, with a D50 of 138 nm and a D90 of 216 nm. A light micrograph using phase optics at 100× of the milled tacrolimus following twelve days of storage under refrigeration is shown in FIG. 9.

The results demonstrate the successful preparation of a stable nanoparticulate tacrolimus formulation, as the mean particle size obtained was 169 nm, and minimal change in particle size was observed following storage.

### Example 6

The purpose of this example was to prepare a nanoparticulate tacrolimus formulation.

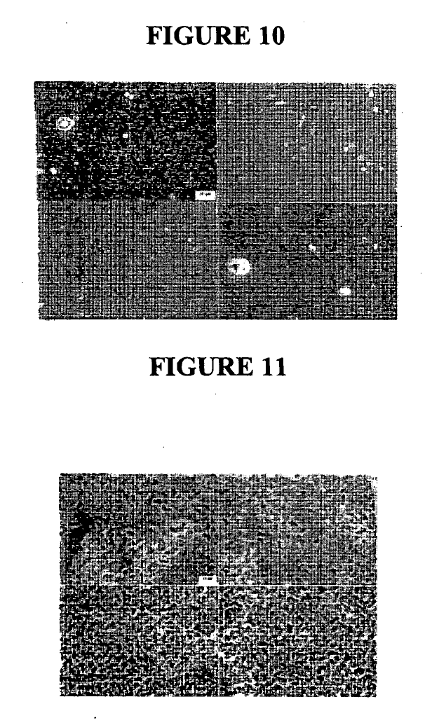

An aqueous dispersion of 5% (w/w) tacrolimus (Camida LLC), combined with 1% (w/w) HPC-SL and 0.1% (w/w) sodium deoxycholate, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 5500 rpms for 75 minutes. A light micrograph using phase optics at 100× of the milled tacrolimus is shown in FIG. 10.

Following milling, the particle size of the milled tacrolimus particles was measured, in deionized distilled water, using a Horiba LA 910 particle size analyzer. The initial mean milled tacrolimus particle size was 1,780 nm, with a D50 of 220 nm and a D90 of 6,665 nm. In a second measurement in distilled water following 12 days of refrigeration at <15° C., the mean tacrolimus particle size was 65,100 nm, with a D50 of 31,252 nm and a D90 of 175,813 nm. A light micrograph using phase optics at 100× of the milled tacrolimus following twelve days of storage under refrigeration is shown in FIG. 11.

The results demonstrate the unsuccessful preparation of a stable nanoparticulate tacrolimus formulation, as significant particle size growth and agglomeration were observed following twelve days of storage. Moreover, the light micrograph using phase optics at 100× following milling also shows the presence of large, possible “unmilled” crystals.

### Example 7

The purpose of this example was to prepare a nanoparticulate tacrolimus formulation.

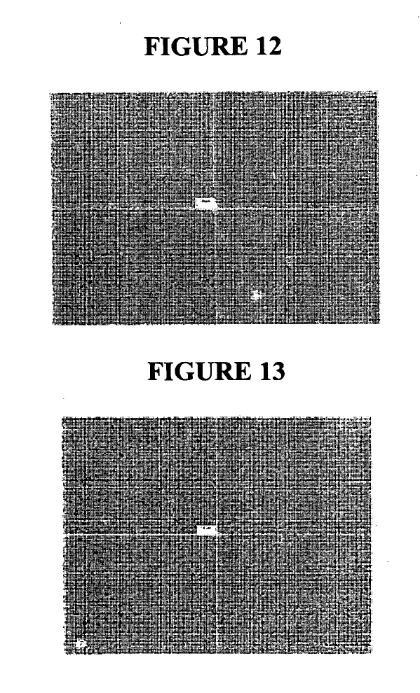

An aqueous dispersion of 10% (w/w) tacrolimus (Camida LLC) combined with 2% (w/w) hydroxypropylmethylcellulose (HPMC) and 0.05% (w/w) DOSS, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes. A light micrograph using phase optics at 100× of the milled tacrolimus is shown in FIG. 12.

Following milling, the particle size of the milled tacrolimus particles was measured, in deionized distilled water, using a Horiba LA 910 particle size analyzer. The initial mean milled tacrolimus particle size was 215 nm, with a D50 of 196 nm and a D90 of 311 nm. In a second measurement in distilled water following 1 week of refrigeration at <15° C., the mean tacrolimus particle size was 227 nm, with a D50 of 206 nm and a D90 of 337 nm. A light micrograph using phase optics at 100× of the milled tacrolimus following one week of storage under refrigeration is shown in FIG. 13.

The results demonstrate the successful preparation of a stable nanoparticulate tacrolimus formulation, as the mean particle size obtained was 215 nm, and minimal particle size growth was observed following storage.

### Example 8

The purpose of this example was to prepare a nanoparticulate tacrolimus formulation.

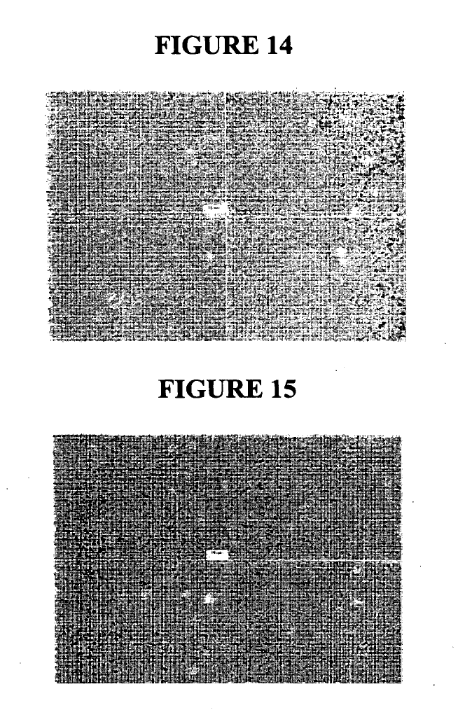

An aqueous dispersion of 10% (w/w) tacrolimus (Camida LLC) and 2% (w/w) Pluronic® F108 was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes. A light micrograph using phase optics at 100× of the milled tacrolimus is shown in FIG. 14.

Following milling, the particle size of the milled tacrolimus particles was measured, in deionized distilled water, using a Horiba LA 910 particle size analyzer. The initial mean milled tacrolimus particle size was 237 nm, with a D50 of 212 nm and a D90 of 355 nm. In a second measurement in distilled water following 1 week of refrigeration at <15° C., the mean tacrolimus particle size was 332 nm, with a D50 of 306 nm and a D90 of 467 nm. A light micrograph using phase optics at 100× of the milled tacrolimus following one week of storage under refrigeration is shown in FIG. 15.

The results demonstrate the successful preparation of a stable nanoparticulate tacrolimus formulation, as the mean particle size obtained was 237 nm, and minimal particle size growth was observed following storage.

### Example 9

The purpose of this example was to prepare a nanoparticulate tacrolimus formulation.

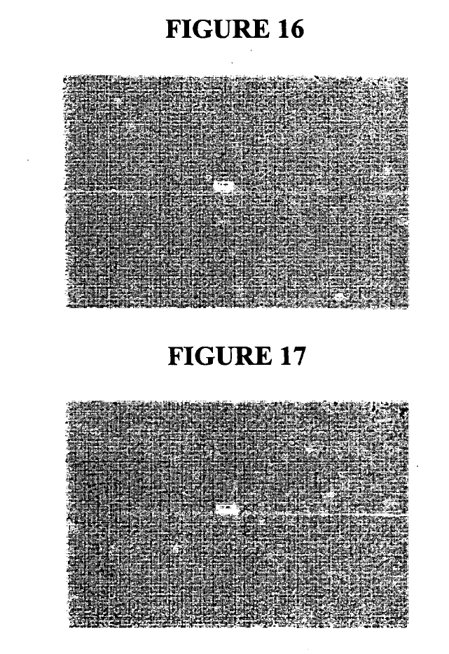

An aqueous dispersion of 10% (w/w) tacrolimus (Camida LLC) and 2% (w/w) Tween® 80 was milled in a 10 ml chamber of a NanoMill® 0.001 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes. A light micrograph using phase optics at 100× of the milled tacrolimus is shown in FIG. 16.

Following milling, the particle size of the milled tacrolimus particles was measured, in deionized distilled water, using a Horiba LA 910 particle size analyzer. The initial mean milled tacrolimus particle size was 208 nm, with a D50 of 191 nm and a D90 of 298 nm. In a second measurement in distilled water following 1 week of refrigeration at <15° C., the mean tacrolimus particle size was 406 nm, with a D50 of 348 nm and a D90 of 658 nm. A light micrograph using phase optics at 100× of the milled tacrolimus following one week of storage under refrigeration is shown in FIG. 17.

The results demonstrate that this formulation is probably not preferred, as the tacrolimus particle size almost doubled after one week of storage.

1. A nanoparticulate dispersion of tacrolimus comprising:
(a) particles of tacrolimus having an effective average particle size of less than about 2000 nm; and
(b) at least one surface stabilizer.

2. The nanoparticulate dispersion of claim 1, wherein the tacrolimus is selected from the group consisting of a crystalline phase, an amorphous phase, and a semi-crystalline phase.

3. The nanoparticulate dispersion of claim 1, wherein the effective average particle size of the nanoparticulate tacrolimus particles is selected from the group consisting of less than about 1900 nm, less than about 1800 nm, less than about 1700 mm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

4. The nanoparticulate dispersion of claim 1, wherein the nanoparticulate dispersion is formulated:
(a) for administration selected from the group consisting of oral, pulmonary, rectal, ophthalmic, colonic, parenteral, intracisternal, intravaginal, intraperitoneal, local, buccal, nasal, and topical administration;
(b) into a dosage form selected from the group consisting of liquid dispersions, solid dispersions, liquid-filled capsule, gels, aerosols, ointments, creams, lyophilized formulations, tablets, capsules, multi-particulate filled capsule, tablet composed of multi-particulates, compressed tablet, and a capsule filled with enteric-coated beads of tacrolimus,
(c) into a dosage form selected from the group consisting of controlled release formulations, fast melt formulations, delayed release formulations, extended release formulations, pulsatile release formulations, and mixed immediate release and controlled release formulations; or
(d) any combination of (a), (b), and (c).

5. The nanoparticulate dispersion of claim 4 formulated for injectable administration, wherein the tacrolimus has an effective average particle size of less than about 600 nm.

6. The nanoparticulate dispersion of claim 5, comprising as a surface stabilizer a povidone polymer having a molecular weight of about 40,000 daltons or less.

7. The nanoparticulate dispersion of claim 1, which is an enteric-coated formulation of nanoparticulate tacrolimus.

8. The nanoparticulate enteric-coated formulation of claim 7, wherein the formulation reduces or eliminates the nausea and vomiting associated with oral administration of non-nanoparticulate or solubilized tacrolimus.

9. The nanoparticulate dispersion of claim 1, wherein the nanoparticulate dispersion further comprises one or more pharmaceutically acceptable excipients, carriers, or a combination thereof.

10. The nanoparticulate dispersion of claim 1, wherein the tacrolimus is present in an amount selected from the group consisting of from about 99.5% to about 0.001%, from about 95% to about 0.1%, and from about 90% to about 0.5%, by weight, based on the total combined weight of the tacrolimus and at least one surface stabilizer, not including other excipients.

11. The nanoparticulate dispersion of claim 1, wherein the at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the tacrolimus and at least one surface stabilizer, not including other excipients.

12. The nanoparticulate dispersion of claim 1, comprising at least one primary surface stabilizer and at least one secondary surface stabilizer.

13. The nanoparticulate dispersion of claim 1, wherein the surface stabilizer is selected from the group consisting of an anionic surface stabilizer, a cationic surface stabilizer, a zwitterionic surface stabilizer, a non-ionic surface stabilizer, and an ionic surface stabilizer.

14. The nanoparticulate dispersion of claim 1, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-p-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-p-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, polyquarternium 10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, quaternized ammonium salt polymers, alkyl pyridinium salts; amines, amine salts, amine oxides, imide azolinium salts, protonated quaternary acrylamides, methylated quaternary polymers, and cationic guar.

15. The nanoparticulate dispersion of claim 1, additionally comprising one or more non-tacrolimus active agents.

16. The nanoparticulate dispersion of claim 1, wherein upon administration to a mammal the tacrolimus particles redisperse such that the particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

17. The nanoparticulate dispersion of claim 1, wherein the nanoparticulate dispersion redisperses in a biorelevant media such that the tacrolimus particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 mm, less than about 600 nm, less than about 550 nm, less than about 500 nm, less than about 450 nm, less than about 400 nm, less than about 350 mm, less than about 300 nm, less than about 250 nm, less than about 200 nm, less than about 150 mm, less than about 100 mm, less than about 75 nm, and less than about 50 nm.

18. The nanoparticulate dispersion of claim 17, wherein the biorelevant media is selected from the group consisting of water, aqueous electrolyte solutions, aqueous solutions of a salt, aqueous solutions of an acid, aqueous solutions of a base, and combinations thereof.

19. The nanoparticulate dispersion of claim 1, wherein the Tₘₐₓ of the tacrolimus, when assayed in the plasma of a mammalian subject following administration, is less than the Tₘₐₓ for a non-nanoparticulate tacrolimus formulation, administered at the same dosage.

20. The nanoparticulate dispersion of claim 19, wherein the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate tacrolimus formulation, administered at the same dosage.

21. The nanoparticulate dispersion of claim 19, wherein the nanoparticulate dispersion exhibits a Tₘₐₓ selected from the group consisting of less than about 6 hours, less than about 5 hours, less than about 4 hours, less than about 3 hours, less than about 2 hours, less than about 1 hour, and less than about 30 minutes after administration to fasting subjects.

22. The nanoparticulate dispersion of claim 1, wherein the Cₘₐₓ of the tacrolimus, when assayed in the plasma of a mammalian subject following administration, is greater than the Cₘₐₓ for a non-nanoparticulate tacrolimus formulation, administered at the same dosage.

23. The nanoparticulate dispersion of claim 22, wherein the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate formulation of tacrolimus, administered at the same dosage.

24. The nanoparticulate dispersion of claim 1, wherein the AUC of the tacrolimus, when assayed in the plasma of a mammalian subject following administration, is greater than the AUC for a non-nanoparticulate tacrolimus formulation, administered at the same dosage.

25. The nanoparticulate dispersion of claim 24, wherein the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of tacrolimus, administered at the same dosage.

26. The nanoparticulate dispersion of claim 1 which does not produce significantly different absorption levels when administered under fed as compared to fasting conditions.

27. The nanoparticulate dispersion of claim 26, wherein the difference in absorption of the tacrolimus nanoparticulate dispersion of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

28. The nanoparticulate dispersion of claim 1, wherein administration of the nanoparticulate dispersion to a human in a fasted state is bioequivalent to administration of the nanoparticulate dispersion to a subject in a fed state.

29. The nanoparticulate dispersion of claim 28, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

30. A method of making a tacrolimus composition comprising contacting particles of tacrolimus with at least one surface stabilizer for a time and under conditions sufficient to provide a tacrolimus composition having an effective average particle size of less than about 2000 nm.

31. The method of claim 30, wherein the contacting comprises grinding, wet grinding, homogenizing, or precipitation.

32. The method of claim 30, wherein the effective average particle size of the tacrolimus particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 650 nm, less than about 600 nm, less than about 550 nm, less than about 500 nm, less than about 450 mm, less than about 400 nm, less than about 350 nm, less than about 300 nm, less than about 250 nm, less than about 200 nm, less than about 150 nm, less than about 100 nm, less than about 75 nm, and less than about 50 nm.

33. A method for the prophylactic treatment of organ rejection comprising administering to a subject in need an effective amount of a tacrolimus composition comprising:
(a) particles of tacrolimus having an effective average particle size of less than about 2000 nm; and
(b) at least one surface stabilizer.

34. The method of claim 33, wherein the subject is a human.

35. The method of claim 33, wherein:
(a) the tacrolimus composition is injectable; and
(b) the effective average particle size of the tacrolimus particles is less than about 600 nm.

36. The method of claim 35, wherein the surface stabilizer is a povidone polymer having a molecular weight of 40,000 daltons or less.

37. The method of claim 33, wherein the tacrolimus composition is enteric-coated.

38. The method of claim 37, wherein the enteric-coated tacrolimus composition is formulated to provide controlled release of tacrolimus in vivo such that only a single dosage per day is required to maintain therapeutic blood concentrations of tacrolimus.
